(19) United States
(12) Patent Application Publication
(10) Pub. No.: US 2006/0189565 A1
(43) Pub. Date: Aug. 24, 2006

(54) Pharmaceutical compositions of ganciclovir

(76) Inventors: Rajeev Shankar Mathur, Delhi (IN); Pananchukunnath Manoj Kumar, New Delhi (IN); Sunilendu Bhushan Roy, Maharashtra (IN); Rajiv Malik, Vienna (AT)

Correspondence Address: RANBAXY INC., 600 COLLEGE ROAD EAST, SUITE 2100, PRINCETON, NJ, 08540, US

(21) Appl. No.: 10/532,024

(22) PCT Filed: Oct. 22, 2003
(86) PCT No.: PCT/IB03/04664

§ 371 Date: Apr. 7, 2006

(30) Foreign Application Priority Data
Oct. 22, 2002 (IN) 1058/DEL/2002

(51) Int. Cl. A61K 31/7072

(52) U.S. Cl. 514/49

(57) ABSTRACT

The technical field of the invention relates to pharmaceutical compositions of 9-(1,3-dihydroxy-2-propoxymethyl) guanine (ganciclovir) that are stable and contain more than 1% water content. One pharmaceutical composition includes ganciclovir having more than about 1% water content, and one or more pharmaceutically acceptable excipients. The ganciclovir retains at least about 97% of its initial purity after one month, at least about 96% of its initial purity after two months, and at least about 95% of its initial purity after three months when stored at 40° C. and 75% RH. In particular, the water content may be between about 2% and about 6%.

### TECHNICAL FIELD OF THE INVENTION

The technical field of the invention relates to pharmaceutical compositions of 9-(1,3-dihydroxy-2-propoxymethyl) guanine that are stable and contain more than 1% water content.

### BACKGROUND OF THE INVENTION

9-(1,3-dihydroxy-2-propoxymethyl) guanine, commonly known as ganciclovir, is a well-known anti-viral agent. It is an acyclic nucleoside analogue of 2′-deoxy guanosine that inhibits replication of herpes virus. Ganciclovir has been shown to be active against cytomegalovirus (CMV) and herpes simplex virus (HSV) in human clinical studies.

U.S. Pat. No. 4,199,574 discloses ganciclovir generically. Ganciclovir and its salts having anti-viral activity were first disclosed in U.S. Pat. No. 4,355,032, assigned to Syntex Inc. The '032 patent describes the preparation of ganciclovir and also outlines the manufacture of pharmaceutical dosage forms containing ganciclovir.

In a subsequent patent, U.S. Pat. No. 4,642,346, assignee Syntex Inc. disclosed a stable anhydrous crystalline form of ganciclovir containing less than 1% water of hydration. The '346 patent states that “anhydrous” refers to a crystalline form which contains less than 1% water of hydration. The '346 patent also states that the earlier disclosed form was reported to be unstable as a result of its hygroscopic nature, which results in handling and formulating problems. The anhydrous form has been shown to be unusually resistant to water absorption, have better physical characteristics than the known hydrate form, and retain a better physical appearance over a longer period of time. Enhancing the appearance of the dosage form increases consumer and physician acceptance.

### SUMMARY OF THE INVENTION

In one general aspect there is provided a pharmaceutical composition that includes ganciclovir having more than about 1% water content; and one or more pharmaceutically acceptable excipients. The ganciclovir retains at least about 97% of its initial purity after one month, at least about 96% of its initial purity after two months, and at least about 95% of its initial purity after three months when stored at 40° C. and 75% RH.

Embodiments of the pharmaceutical composition may include one or more of the following features. For example, the water content (water of hydration) may be more than about 1.5%, may be between about 0.1% and about 10%, and, more particularly, may be from about 2% to about 6%.

The one or more pharmaceutically acceptable excipients may include one or more of diluents, binding agents, disintegrants, wetting agents, lubricants, glidants, and anti-adherent agents. The diluent may include one or more of lactose, starch, mannitol, sorbitol, dextrose monohydrate, microcrystalline cellulose, dibasic calcium phosphate dihydrate, sucrose-based diluents, monobasic calcium sulphate monohydrate, calcium sulphate dihydrate, calcium lactate trihydrate, and powdered cellulose.

The binding agent may be one or more of acacia, tragacanth, sucrose, gelatin, glucose, starch, alginic acid, polyethylene glycol, guar gum, polysaccharides, bentonites, polyvinylpyrrolidone, cellulose ethers, hydroxypropyl methylcellulose, and hydroxypropyl cellulose. The binding agent may make up between approximately 0.05% and approximately 5% w/w of the composition.

The disintegrant may be one or more of starches, sodium starch glycolate, clays, celluloses, purified cellulose, methylcellulose, sodium carboxymethylcellulose, alginates, pre-gelatinized corn starches, crospovidone, and gums. The disintegrant may make up between approximately 0.5% and approximately 7% w/w of the composition. A portion of the disintegrant may be present extragranularly. The extragranular disintegrant may be between approximately 0.5% and approximately 3% w/w of the composition.

The pharmaceutical composition may include between approximately 80% and approximately 90% w/w ganciclovir, between approximately 6% and approximately 8% w/w microcrystalline cellulose, between approximately 2% and approximately 4% w/w povidone, between approximately 2.5% and approximately 5% w/w croscarmellose sodium, and between approximately 0.25% and 0.75% w/w magnesium stearate. Approximately half of the croscarmellose sodium may be present extragranularly and the other half may be present intragranularly.

In another general aspect, there is provided a process for the preparation of a pharmaceutical composition that includes ganciclovir having a water content of more than about 1% and one or more pharmaceutically acceptable excipients. The process includes

a) blending the ganciclovir having a water content of more than 1% with the one or more pharmaceutically acceptable excipients;

b) optionally granulating the blend by wet granulation or dry granulation;

c) lubricating the blend of step a) or the granules of step b); and

d) compressing into or filling into a solid dosage form.

The ganciclovir retains at least about 97% of its initial purity after one month, at least about 96% of its initial purity after two months, and at least about 95% of its initial purity after three months when stored at 40° C. and 75% RH.

Embodiments of the process may include one or more of the following features. For example, the water content may be more than about 1.5%, may be between about 1% and about 10%, and, more particularly, may be from about 2% to about 6%.

The one or more pharmaceutically acceptable excipients may be one or more of diluents, binding agents, disintegrants, wetting agents, lubricants, glidants, and anti-adherent agents. The diluent may be one or more of lactose, starch, mannitol, sorbitol, dextrose monohydrate, microcrystalline cellulose, dibasic calcium phosphate dihydrate, sucrose-based diluents, monobasic calcium sulphate monohydrate, calcium sulphate dihydrate, calcium lactate trihydrate, and powdered cellulose.

The binding agent may be one or more of acacia, tragacanth, sucrose, gelatin, glucose, starch, alginic acid, polyethylene glycol, guar gum, polysaccharides, bentonites, polyvinylpyrrolidone, cellulose ethers, hydroxypropyl methylcellulose, and hydroxypropyl cellulose. The binding agent may make up between about 0.05% and about 5% w/w of the composition.

The disintegrant may be one or more of starches, sodium starch glycolate, clays, celluloses, purified cellulose, methylcellulose, sodium carboxymethylcellulose, alginates, pre-gelatinized corn starches, crospovidone, and gums. The disintegrant may make up between about 0.5% and about 7% w/w of the composition. A portion of the disintegrant may be extragranular. The extragranular disintegrant may make up between about 0.5% and about 3% w/w of the formulation.

The granules may be filled into a capsule or compressed into a tablet. The granules after the granulation process may have a bulk density of at least 0.6 g/ml. The granules after the granulation process may have a tapped density of less than 0.8 g/ml.

The wet granulation may include granulating the ganciclovir and one or more pharmaceutically acceptable excipients with a binder solution; drying the granules; mixing the dried granules with one or more extragranular excipients; and compressing the resultant blend into a tablet or filling into a capsule.

The dry granulation may include dry compaction of the ganciclovir with the one or more pharmaceutically acceptable excipients; breaking the compacts to generate granules; mixing the granules with one or more extragranular excipients; and compressing the resultant blend into a tablet or filling into a capsule.

In another general aspect there is provided a method of treating infection caused by one or both of cytomegalovirus and herpes simplex virus by administering a pharmaceutical composition to a patient in need thereof. The pharmaceutical composition includes ganciclovir having more than about 1% water content and one or more pharmaceutically acceptable excipients. The ganciclovir retains at least about 97% of its initial purity after one month, at least about 96% of its initial purity after two months, and at least about 95% of its initial purity after three months when stored at 40° C. and 75% RH.

Embodiments of the method may include one or more of the features described above. For example, the water content may be more than about 1.5%, may be between about 1% and about 10%, and, more particularly, may be from about 2% to about 6%.

In another general aspect there is provided a ganciclovir capsule for oral administration. The ganciclovir capsule includes ganciclovir having between about 2% and about 6% water content; between approximately 80% and approximately 90% w/w ganciclovir; between approximately 6% and approximately 8% w/w microcrystalline cellulose; between approximately 2% and approximately 4% w/w povidone; between approximately 2.5% and approximately 5% w/w croscarmellose sodium; and between approximately 0.25% and 0.75% w/w magnesium stearate, The ganciclovir retains at least about 97% of its initial purity after one month, at least about 96% of its initial purity after two months, and at least about 95% of its initial purity after three months when stored at 40° C. and 75% RH.

Embodiments of the gancicloivr capsule may include one or more of the features described above. For example, approximately half of the croscarmellose sodium may be present extragranularly and the other half may be present intragranularly.

The details of one or more embodiments of the inventions are set forth in the description below. Other features, objects and advantages of the inventions will be apparent from the description and claims.

### DETAILED DESCRIPTION OF THE INVENTION

Ganciclovir is a high dose drug and, therefore, drug characteristics such as stability play an important role in determining the characteristics of the final formulation. The inventors have surprisingly found that it is possible to prepare a stable pharmaceutical formulation that includes ganciclovir containing more than 1% water content. In particular, the inventors have shown that even when the active ingredient used in the formulation is ganciclovir having more than 1% water content instead of the anhydrous crystalline ganciclovir disclosed in the prior art, the ganciclovir does not absorb substantial amounts of moisture, cause handling problems, or cause formulating problems. Furthermore, the formulation exhibited acceptable stability in spite of containing ganciclovir with a water content of more than 1%.

Without intending to be limited by theories, the inventors believe that the water content helped in binding of the drug and excipients thereby helping in formulating. For example, in one of the embodiments, a stable pharmaceutical formulation includes ganciclovir having more than 1% water content. In another embodiment, a stable pharmaceutical formulation includes ganciclovir containing more than 1.5% water content. In yet another embodiment, a stable pharmaceutical formulation includes ganciclovir containing about 1% to about 10% water content, or from about 2% to about 6%.

The pharmaceutical compositions and dosage forms described herein can be administered to an individual in need of the composition or dosage form for treating an infection caused by cytomegalovirus (CMV) and/or herpes simplex virus (HSV) by administering stable pharmaceutical composition that contains ganciclovir containing more than 1% water content and is nonetheless stable. In particular, the ganciclovir can contain about 1% to about 10% water content.

The invention further includes a process of preparing a solid unit dosage form that includes ganciclovir having 1% or more water content. In general, the process includes granulating ganciclovir or a pharmaceutically acceptable salt thereof, according to the methods known in the art, followed by compression of the granules into a tablet or filling them into a hard gelatin capsule. In particular, a process for preparing the stable ganciclovir pharmaceutical compositions described herein includes the steps of:
a) blending ganciclovir having a water content of more than about 1% with one or more pharmaceutically acceptable excipients,
b) optionally granulating the blend by wet granulation or dry granulation as herein described,
c) lubricating the blend of step a) or granules of step b), and
d) compressing into or filling into a suitable size solid dosage form.

Ganciclovir may be granulated with the pharmaceutically excipients using any of the conventional methods used in the art including wet granulation, dry granulation, and direct compression. In the wet granulation method, the dry solids (active ingredients, filler, disintegrant, etc.) are blended and moistened with the binder solution and then the agglomerates or granules are built up of the moistened solids. Wet massing is continued until a desired homogeneous particle size has been achieved whereupon the granulated product is dried to form dried granules. The dried granules are blended with lubricants and, optionally, a disintegrant and the blend then is compressed into tablets or filled into hard gelatin capsules.

In the dry granulation method, the active ingredient can be compacted alone or together with other pharmaceutically acceptable excipients. The granules then are mixed with extragranular excipients and compressed into tablets or filled into hard gelatin capsules.

Thus, the ganciclovir dosage forms can be a tablet dosage form prepared by compression of granules of active ingredient and pharmaceutically acceptable excipients obtained by the wet granulation method or the dry granulation method. The ganciclovir dosage forms also can be a capsule prepared by filling granules of active ingredient and pharmaceutically acceptable excipients obtained by the wet granulation method or the dry granulation method in a hard gelatin capsule.

The density of the granules as measured by the bulk density and the tapped density is an important parameter for this formulation. The difference between these two densities describes the cohesiveness and compressibility of the substance. These two parameters are particularly important for the capsule dosage form and are used to decide the optimum filling of the capsule. A bulk density of at least 0.6 g/ml and a tapped density of less than 0.8 g/ml are preferred to achieve the optimum filling of the capsules. Bulk density is measured using the procedure described in USP 25, First Annual Asian Edition, 2002, page 1981-1982, the contents of which are incorporated herein by reference. Generally, bulk density is determined by measuring the volume of a known mass of powder sample that has been passed through a screen into a graduated cylinder (Method I of USP).

Tapped density is determined by mechanically tapping a measuring cylinder containing a powder sample. After observing the initial volume, the cylinder is mechanically tapped, and volume readings are taken until a further volume change was observed. The mechanical tapping is achieved by raising the cylinder and then allowing it to drop under its own weight a specified distance. For example, an Electrolab Tap Density apparatus may be used for tapping the cylinder.

The pharmaceutical compositions contain ganciclovir in a desired amount admixed with one or more pharmaceutically acceptable excipients. The pharmaceutically acceptable excipients may be one or more of diluents, disintegrants, binding agents, wetting agents, lubricants, glidants, and anti-adherent agents.

The diluent may be one or more of lactose, starch, mannitol, sorbitol, dextrose monohydrate, microcrystalline cellulose, dibasic calcium phosphate dihydrate, sucrose-based diluents, monobasic calcium sulphate monohydrate, calcium sulphate dihydrate, calcium lactate trihydrate, powdered cellulose, and the like.

The binding agent is selected from those commonly known in the art, and is used to impart sufficient cohesion to the powders to permit normal processing, such as sizing, lubrication, compression, and packaging, but still permit the composition to disintegrate and dissolve upon ingestion. Examples of suitable binding agents include one or more of acacia, tragacanth, sucrose, gelatin, glucose, starch, alginic acid, polyethylene glycol, guar gum, polysaccharides, bentonites, polyvinylpyrrolidone, and cellulose ethers such as hydroxypropyl methylcellulose and hydroxypropyl cellulose. The binding agent preferably is present at from about 0.05% to about 5% w/w of the formulation, although variations outside this range may be used.

The disintegrants may be one or more of starches, sodium starch glycolate, clays, celluloses such as purified cellulose, methylcellulose and sodium carboxymethylcellulose, alginates, pre-gelatinized corn starches, crospovidone, and gums. Disintegrants can be added at any suitable step during the preparation of the pharmaceutical composition, particularly prior to granulation or during the lubrication step prior to compression or filling of the dosage form. The disintegrant may be present either or both of intragranularly and extragranularly.

Croscarmellose sodium is one preferred disintegrant and may be present at from about 0.5% to about 7% w/w of the formulation. We have observed that use of disintegrant intragranularly as well as extragranularly enhances the disintegration time appreciably. The extragranular disintegrant is present at from about 0.5% to about 3% w/w of the formulation, and preferably the disintegrant or disintegrants are present at about 1.5% to about 2.5% w/w of the formulation.

The pharmaceutical composition optionally comprises one or more lubricants and/or glidants. Suitable lubricants and/or glidants include glyceryl behenate, metallic stearate such as magnesium stearate, stearic acid, hydrogenated vegetable oils, talc, waxes, boric acid, sodium benzoate, polyethylene glycols and sodium stearyl fumarate. The lubricant used in the present formulation is present in an amount of about 0.1% to about 2.0% w/w and preferably from about 0.1% to about 1.5% w/w. Use of magnesium stearate as lubricant is particularly desirable.

Commercial formulations can contain ganciclovir having a water content of from about 2% to about 6% w/w. Three particular unit formulae for ganciclovir 250 mg and 500 mg dosage forms are provided below merely to exemplify the invention but not intended to limit the scope of the invention.

| | | |
|---|---|---|
| Ingredients | Amount (mg) | Percentage (%) |

UNIT FORMULA (I) OF 500 MG GANCICLOVIR DOSAGE FORM

| | | |
|---|---|---|
| Intragranular | | |
| Ganciclovir | 500 | 92.59 |
| Microcrystalline cellulose | 0.45 | 0.083 |
| Polyvinyl pyrrolidone | 16.2 | 3.0 |
| Croscarmellose sodium | 11.0 | 2.04 |
| Extragranular | | |
| Magnesium stearate | 1.35 | 0.25 |
| Croscarmellose sodium | 11.0 | 2.04 |
| Total weight | 540 | 100 |

UNIT FORMULA (II) OF 500 MG GANCICLOVIR DOSAGE FORM

| | | |
|---|---|---|
| Intragranular | | |
| Ganciclovir | 500 | 85.91 |
| Microcrystalline cellulose NF | 41.00 | 7.04 |
| Povidone (K-90) USP | 16.00 | 2.75 |
| Croscarmellose sodium NF | 11.0 | 1.89 |
| Purified water USP | q.s. | — |
| Extragranular | | |
| Magnesium stearate NF | 3.00 | 0.52 |
| Croscarmellose sodium | 11.0 | 1.89 |
| Total capsule fill weight (mg) | 582.00 | 100 |

UNIT FORMULA (III) OF 250 MG GANCICLOVIR DOSAGE FORM

| | | |
|---|---|---|
| Intragranular | | |
| Ganciclovir | 250 | 85.91 |
| Microcrystalline cellulose NF | 20.50 | 7.04 |
| Povidone (K-90) USP | 8.00 | 2.75 |
| Croscarmellose sodium NF | 5.50 | 1.89 |
| Purified water USP | q.s. | — |
| Extragranular | | |
| Magnesium stearate NF | 1.50 | 0.52 |
| Croscarmellose sodium | 5.50 | 1.89 |
| Total capsule fill weight (mg) | 291.00 | 100 |

Exemplary processes for preparing ganciclovir dosage forms are described below but are not intended to, and should not be construed as, limiting the scope of the invention.

### EXAMPLE 1

### Wet Granulation

Ganciclovir is sifted with croscarmellose sodium (intragranular) and microcrystalline cellulose and then granulated with a solution of polyvinyl pyrrolidone in water to form granules. The granules then are dried and blended with magnesium stearate and croscarmellose sodium (extragranular) to form a blend. The blend is filled into a hard gelatin capsule or compressed into a tablet.

### EXAMPLE 2

### Dry Granulation

Ganciclovir is sifted with croscarmellose sodium (intragranular), polyvinyl pyrrolidone, and microcrystalline cellulose to form a first blend. This first blend is compacted and then broken to generate granules. The granules then are mixed with magnesium stearate and croscarmellose sodium (extragranular) to form a second blend. This second blend is filled into a hard gelatin capsule or compressed into a tablet.

Different formulations containing variable percentages of water were subjected to stability and moisture uptake studies. The results of these studies are shown in Table 1 and Table 2. For example, ganciclovir containing 1.99% water content was subjected to moisture uptake at 25° C. and 60% relative humidity (RH) in an open Petri dish and the increase in weight was monitored. The data from this study presented in Table 1 demonstrates there is no appreciable increase in moisture during storage.

TABLE 1
Moisture uptake by ganciclovir

| Time (hrs) | Moisture gain (% w/w) |
|---|---|
| 2.0 | 0.12 |
| 4.0 | 0.38 |
| 8.0 | 0.46 |
| 48.0 | 0.49 |
| 168.0 (1 week) | 0.50 |

Accelerated stability testing was conducted by varying the water content of ganciclovir between 1.99% w/w and 2.54% w/w. The packages of final product were stored at 40° C. and 75% RH for a period of three months. At predetermined intervals, some of the packages were opened and analyzed to determine the amount of active ingredient, related impurities (RS), and water content present in the formulation. The data provided below in Table 2 shows that over the three months of accelerated aging testing at various water contents, the formulation does not pick up a substantial amount of water and remains quite stable.

TABLE 2

| Water content | | | Total RS (%) | |
|---|---|---|---|---|
| of ganciclovir | Storage | | (Except | Water Content |
| (% w/w) | (Months) | Assay (%) | Guanine) | (% w/w) |
| 1.99 | 0 | 97.90 | 0.837 | 5.19 |
| | 1 | 95.96 | 0.849 | 5.02 |
| | 2 | 94.48 | 0.841 | 5.02 |
| | 3 | 94.24 | 0.818 | 5.17 |
| 2.54 | 0 | 102.0 | 0.315 | 4.48 |
| | 1 | 100.7 | 0.335 | 5.12 |
| | 2 | 100.4 | 0.426 | 4.98 |
| | 3 | 99.8 | 0.318 | 4.96 |

As can be seen from Table 2, the ganciclovir is very stable, as illustrated by the assay values reported after storage for three months at 40° C. and 75% RH. Specifically, the ganciclovir loses less than about five percent of its purity after three months, less than about four percent of its purity after two months, and less than about three percent of its purity after one month. In other words, the ganciclovir retains at least about 97% of its initial purity after one month, at least about 96% of its initial purity after two months, and at least about 87% of its initial purity after three months when stored at 40° C. and 75% RH.

While several particular forms of the inventions have been described, it will be apparent that various modifications and combinations of the inventions detailed in the text can be made without departing from the spirit and scope of the inventions. Further, it is comtemplated that any single feature or any combination of optional features of the inventive variations described herein may be specifically excluded from the claimed inventions and be so described as a negative limitation. Accordingly, it is not intended that the inventions be limited, except as by the appended claims.

We claim:

1. A pharmaceutical composition comprising:
ganciclovir having more than about 1% water content; and
one or more pharmaceutically acceptable excipients,
wherein the ganciclovir retains at least about 97% of its initial purity after one month, at least about 96% of its initial purity after two months, and at least about 95% of its initial purity after three months when stored at 40° C. and 75% RH.

2. The pharmaceutical composition according to claim 1, wherein the water content is more than about 1.5%.

3. The pharmaceutical composition according to claim 1, wherein the water content is between about 1% and about 10%.

4. The pharmaceutical composition according to claim 1, wherein the water content is between about 2% and about 6%.

5. The pharmaceutical composition according to claim 1, wherein the one or more pharmaceutically acceptable excipients comprise one or more of diluents, binding agents, disintegrants, wetting agents, lubricants, glidants, and anti-adherent agents.

6. The pharmaceutical composition according to claim 5, wherein the diluent comprises one or more of lactose, starch, mannitol, sorbitol, dextrose monohydrate, microcrystalline cellulose, dibasic calcium phosphate dihydrate, sucrose-based diluents, monobasic calcium sulphate monohydrate, calcium sulphate dihydrate, calcium lactate trihydrate, and powdered cellulose.

7. The pharmaceutical composition according to claim 5, wherein the binding agent comprises one or more of acacia, tragacanth, sucrose, gelatin, glucose, starch, alginic acid, polyethylene glycol, guar gum, polysaccharides, bentonites, polyvinylpyrrolidone, cellulose ethers, hydroxypropyl methylcellulose, and hydroxypropyl cellulose.

8. The pharmaceutical composition according to claim 7, wherein the binding agent comprises between approximately 0.05% and approximately 5% w/w of the composition.

9. The pharmaceutical composition according to claim 6, wherein the disintegrant comprises one or more of starches, sodium starch glycolate, clays, celluloses, purified cellulose, methylcellulose, sodium carboxymethylcellulose, alginates, pre-gelatinized corn starches, crospovidone, and gums.

10. The pharmaceutical composition according to claim 9, wherein the disintegrant comprises between approximately 0.5% and approximately 7% w/w of the composition.

11. The pharmaceutical composition according to claim 10, wherein a portion of the disintegrant is present extragranularly.

12. The pharmaceutical composition according to claim 11, wherein the extragranular disintegrant comprises between approximately 0.5% and approximately 3% w/w of the composition.

13. The pharmaceutical composition according to claim 1, wherein the pharmaceutical composition comprises between approximately 80% and approximately 90% w/w ganciclovir, between approximately 6% and approximately 8% w/w microcrystalline cellulose, between approximately 2% and approximately 4% w/w povidone, between approximately 2.5% and approximately 5% w/w croscarmellose sodium, and between approximately 0.25% and 0.75% w/w magnesium stearate.

14. The pharmaceutical composition according to claim 13, wherein approximately half of the croscarmellose sodium is present extragranularly and the other half is present intragranularly.

15. A process for the preparation of a pharmaceutical composition comprising ganciclovir having a water content of more than about 1% and one or more pharmaceutically acceptable excipients, the process comprising:
a) blending the ganciclovir having a water content of more than 1% with the one or more pharmaceutically acceptable excipients;
b) optionally granulating the blend by wet granulation or dry granulation;
c) lubricating the blend of step a) or the granules of step b); and
d) compressing into or filling into a solid dosage form,
wherein the ganciclovir retains at least about 97% of its initial purity after one month, at least about 96% of its initial purity after two months, and at least about 95% of its initial purity after three months when stored at 40° C. and 75% RH.

16. The process according to claim 15, wherein the water content is more than about 1.5%.

17. The process according to claim 15, wherein the water content is between about 1% and about 10%.

18. The process according to claim 15, wherein the water content is between about 2% and about 6%.

19. The process according to claim 15, wherein the one or more pharmaceutically acceptable excipients comprise one or more of diluents, binding agents, disintegrants, wetting agents, lubricants, glidants, and anti-adherent agents.

20. The process according to claim 19, wherein the diluent comprises one or more of lactose, starch, mannitol, sorbitol, dextrose monohydrate, microcrystalline cellulose, dibasic calcium phosphate dihydrate, sucrose-based diluents, monobasic calcium sulphate monohydrate, calcium sulphate dihydrate, calcium lactate trihydrate, and powdered cellulose.

21. The process according to claim 19, wherein the binding agent comprises one or more of acacia, tragacanth, sucrose, gelatin, glucose, starch, alginic acid, polyethylene glycol, guar gum, polysaccharides, bentonites, polyvinylpyrrolidone, cellulose ethers, hydroxypropyl methylcellulose, and hydroxypropyl cellulose.

22. The process according to claim 21, wherein the binding agent comprises between about 0.05% and about 5% w/w of the composition.

23. The process according to claim 19, wherein the disintegrant comprises one or more of starches, sodium starch glycolate, clays, celluloses, purified cellulose, methylcellulose, sodium carboxymethylcellulose, alginates, pre-gelatinized corn starches, crospovidone, and gums.

24. The process according to claim 23, wherein the disintegrant comprises between about 0.5% and about 7% w/w of the composition.

25. The process according to claim 24, wherein a portion of the disintegrant is extragranular.

26. The process according to claim 25, wherein the extragranular disintegrant comprises between about 0.5% and about 3% w/w of the formulation.

27. The process according to claim 15, wherein the granules are filled into a capsule.

28. The process according to claim 15, wherein the granules are compressed into a tablet.

29. The process according to claim 15, wherein the granules after the granulation process have a bulk density of at least 0.6 g/ml.

30. The process according to claim 15, wherein the granules after the granulation process have a tapped density of less than 0.8 g/ml.

31. The process according to claim 15, wherein the wet granulation comprises:
granulating the ganciclovir and one or more pharmaceutically acceptable excipients with a binder solution;
drying the granules;
mixing the dried granules with one or more extragranular excipients; and
compressing the resultant blend into a tablet or filling into a capsule.

32. The process according to claim 15, wherein the dry granulation comprises:
dry compaction of the ganciclovir with the one or more pharmaceutically acceptable excipients;
breaking the compacts to generate granules;
mixing the granules with one or more extragranular excipients; and
compressing the resultant blend into a tablet or filling into a capsule.

33. A method of treating infection caused by one or both of cytomegalovirus and herpes simplex virus by administering a pharmaceutical composition to a patient in need thereof, the pharmaceutical composition comprising ganciclovir having more than about 1% water content and one or more pharmaceutically acceptable excipients,
wherein the ganciclovir retains at least about 97% of its initial purity after one month, at least about 96% of its initial purity after two months, and at least about 95% of its initial purity after three months when stored at 40° C. and 75% RH.

34. The method of treating of claim 33, wherein the water content of ganciclovir is more than about 1.5%.

35. The method of treating of claim 33, wherein the water content of ganciclovir is between about 1% and about 10%.

36. The method of treating of claim 33, wherein the water content of ganciclovir is between about 2% and about 6%.

37. A ganciclovir capsule for oral administration, the ganciclovir capsule comprising:
ganciclovir having between about 2% and about 6% water content;
between approximately 80% and approximately 90% w/w ganciclovir;
between approximately 6% and approximately 8% w/w microcrystalline cellulose;
between approximately 2% and approximately 4% w/w povidone;
between approximately 2.5% and approximately 5% w/w croscarmellose sodium; and
between approximately 0.25% and 0.75% w/w magnesium stearate,
wherein the ganciclovir retains at least about 97% of its initial purity after one month, at least about 96% of its initial purity after two months, and at least about 95% of its initial purity after three months when stored at 40° C. and 75% RH.

38. The ganciclovir capsule according to claim 37, wherein approximately half of the croscarmellose sodium is present extragranularly and the other half is present intragranularly.
